(19) United States
(12) Patent Application Publication
(10) Pub. No.: US 2018/0028419 A1
(43) Pub. Date: Feb. 1, 2018

(54) DEPILATORY COMPOSITION

(71) Applicant: Sofibel S.A.S., Levallois Perret (FR)

(72) Inventor: Amina Saaid, Rieux (FR)

(73) Assignee: Sofibel S.A.S., Levallois Perret (FR)

(21) Appl. No.: 15/658,827

(22) Filed: Jul. 25, 2017

(30) Foreign Application Priority Data
Jul. 29, 2016 (EP) 16182026.1

(51) Int. Cl. A61K 8/20; A61Q 9/04; A61K 8/31; A45D 26/00; A61K 8/37; A61K 8/81; A61K 8/27; A61K 8/60; A61K 8/92

(52) U.S. Cl. CPC A61K 8/20; A61K 8/60; A61Q 9/04; A61K 8/31; A61K 8/92; A61K 8/37; A61K 8/8111; A61K 8/8152; A61K 8/27; A45D 26/0014; A61K 2800/54; A61K 2800/87; A61K 2800/805

(57) ABSTRACT

The invention provides a depilatory composition comprising at least one rosinous or resinous tackifying agent, at least one surfactant, and at least a metal salt as a heating improvement agent. The invention provides an enhanced heating profile of the depilatory composition.

### FIELD OF THE INVENTION

The present invention relates to a depilatory composition, and to its use for removal of unwanted hair from humans.

### TECHNICAL BACKGROUND

Depilatory compositions formed of viscoelastic materials are well known. Known viscoelastic materials include those which are rosin-based. In some products the depilatory compositions may be supplied in the form of strips, retained between cellophane or non-woven sheets. In use, the user peels away one of the sheets, presses the depilatory strip firmly onto the area to be plucked, then pulls one end of the remaining cellophane sheet sharply away from the area.

The hairs trapped in the composition are removed from the treated area along with, optimally, all of the composition, still attached to the remaining backing strip.

In one approach a composition may be warmed and applied to the skin, for example by means of a spatula or other applicator. The composition is then allowed to set or harden and then pulled sharply to remove the depilatory material and hair from the skin without the necessity of applying a strip of fabric.

It is often a requirement that the composition be first warmed. Warming can be conducted in a variety of ways, for example by placing a container containing the composition in a pan of hot water, into a microwave oven or into an appropriate electrical heating device. A potential problem with such warming methods is that the heating device may not heat the composition consistently throughout and one or more hot regions or hot spots may develop or the product itself may become overheated.

An exemplary depilatory composition is described in EP1245220, which discloses a depilatory composition comprising a rosinous material, water and a surfactant. Another exemplary depilatory composition is described in WO2005/112876, which discloses a depilatory composition comprising a rosinous material, wax, an elastomer, a surfactant and water. A particular product named “cire professionnelle, jambes-maillot-aisselles” is distributed by Groupe Auchan SA and, as indicated on its promotional material, the product has the following composition: glyceryl rosinate, cera alba, paraffin, peg40, hydrogenated castor oil, hydrogenated styrene-butadiene copolymer, aqua, parfum, ethoxydiglycol, glucose, lactic acid, chamomilla recutita flower extract, hexyl cinnamal, caramel, sodium benzoate, linalool, coumarin, potassium sorbate, hydroxyisohexyl 3-cyclohexene carboxaldehyde, alpha-isomethyl ionone, bisabolol, geraniol, limonene, benzvl alcohol, CI 77019, CI 77891. The Applicant has also distributed a product named “bye bye douleur”, which is water-free product comprising glyceryl rosinate, triethylene glycol rosinate, paraffin, hydrogenated styrene/butadiene copolymer, cera microcristallina, zinc oxide and mica. As a further example of depilatory compositions, WO-A-2013/160690 discloses a depilatory composition which comprises exhibits a thermal heat conductivity of from 50 to 2000, preferably from 200 to 400, Wm⁻¹K⁻¹, which is achieved by adding a required amount of a thermal interface material selected from the group consisting of aluminium-diamond, aluminium-graphite fibres, aluminium nitride, aluminium oxide, beryllium oxide, boron carbide, boron nitride, copper-graphite fibres, gold, gold coated powders, graphite, iron oxide, magnesium oxide, nickel gold additives, nickel graphite, nickel powders, silicon carbide, silicon nitride, silver, silver coated powders, silver-diamond, tin oxide, titanium carbide, titanium dioxide and zinc oxide or combinations thereof, preferably boron nitride.

### SUMMARY OF THE INVENTION

The invention provides a depilatory composition comprising at least one rosinous or resinous tackifying agent, at least one surfactant, water and a metal salt as heating improving agent.

The invention provides one or more of the following advantages:
the heating profile is enhanced, with a more uniform temperature throughout; and
the composition has an improved viscosity profile, with a low viscosity achieved at a lower temperature, and a more creamy feeling, especially when a thermoplastic and/or elastomeric polymer is used in the composition.

According to one embodiment the metal salt is selected from the group consisting of halides or polyatomic oxygen-containing negative ion of alkaline or alkaline-earth metals. Preferably the metal salt heating improving agent is NaCl.

According to one embodiment, the amount of metal salt is about 0.1% to about 10% by weight, preferably about 0.25% to about 5% by weight, more preferably about 0.5 to about 2% by weight, of the total weight of the depilatory composition.

According to one embodiment, the depilatory composition further comprises:
at least one texturizing agent, preferably selected from the group consisting of an oil, a fatty acid ester, a wax and mixtures thereof.

According to one embodiment, the depilatory composition further comprises:
at least one polymer, preferably a thermoplastic polymer or an elastomeric polymer.

According to one embodiment, the depilatory composition comprises based on the total weight of the depilatory composition:
about 50 to about 90% by weight, preferably about 60 to about 80% by weight, of at least one rosinous or resinous tackifying agent, based on the total weight of the depilatory composition;
about 7 to about 30% by weight, preferably about 15 to about 20% by weight, of at least one texturizing agent selected from the group consisting of an oil, a fatty acid ester, a wax and mixtures thereof, based on the total weight of the depilatory composition;
about 1 to about 15%, preferably about 2 to about 6% by weight, of at least one thermoplastic or elastomeric polymer, based on the total weight of the depilatory composition;
about 1 to about 20% by weight, preferably about 2 to about 8% by weight, of at least one surfactant, based on the total weight of the depilatory composition;
about 0.1 to about 10% by weight, preferably about 0.2 to about 8% by weight of at least one metal salt, based on the total weight of the depilatory composition;
about 3 to about 15% by weight, preferably from about 3 to about 6% by weight, of water, based on the total weight of the depilatory composition.

According to one embodiment, the rosinous or resinous tackifying agent comprises glyceryl rosinate and methyl hydrogenated rosinate. In other embodiments, synthetic resins can be used.

According to one embodiment, the texturizing agent is a wax, preferably selected from the group consisting of paraffin, beeswax, microcrystalline wax and mixtures of two or more thereof, more preferably a mixture of paraffin and microcrystalline wax.

According to one embodiment, the thermoplastic polymer is polyethylene and/or polyalphaolefins (PAO) and the elastomeric polymer is Ethylene Butyl Acrylate (EBA).

According to one embodiment, the surfactant is polysorbate, preferably polysorbate 80.

According to one embodiment, the depilatory composition comprises, based on the total weight of the depilatory composition:
about 60 to about 80% by weight of glyceryl rosinate, and/or methyl hydrogenated rosinate;
about 15 to about 20% by weight of paraffin, and/or microcrystalline wax;
about 2 to about 6% by weight of polyethylene and/or PAO and/or EBA;
about 2 to about 8% by weight of polysorbate;
about 0.5 to about 2% by weight of NaCl;
about 3 to about 6% by weight of water.

The invention also provides for a microwaveable container comprising the depilatory composition according to the invention.

The invention also provides for a method for removing hair from skin which comprises:

(a) heating a depilatory composition according to the invention;

(b) applying said composition to the skin;

(c) allowing said composition to set and hold said hair or applying a strip of material over said composition and allowing said strip to adhere thereto; and;

(d) removing said composition and hair by peeling said composition or said material and said composition, from the skin.

According to one embodiment, said composition is heated by microwaves.

The invention further provides advantages as will be apparent from the description.

### DESCRIPTION OF EMBODIMENTS OF THE INVENTION

The invention is now disclosed in more details in the following description. Unless specified otherwise, amounts and percentages are by weight.

### Rosinous or Resinous Tackifying Agent.

The tackifying agent can be a rosinous or resinous material that is suitable for application to human skin. Natural rosins, natural resins, or synthetic resins may be used. In some embodiments, any one or more of glyceryl rosinate, triethylene glycol rosinate, and methyl hydrogenated rosinate can be used. A preferred combination can be glyceryl rosinate and triethylene glycol rosinate. In such combination, glyceryl rosinate preferably represents the majority of the rosinous or resinous tackifying agent. For example, in combination, glyceryl rosinate can be present in an amount that is about 5 to 20 times and preferably 7 to 10 times more than the triethylene glycol. Such ratios may apply to combinations of glyceryl rosinate with other partner components, such as methyl hydrogenated rosinate or another resinous material or resinous material.

The overall amount of the resinous or resinous tackifying material in the overall depilatory composition preferably is about 50% to 90% by weight, more preferably about 60% to 80% by weight based on the total weight of the depilatory composition.

### Texturizing Agent

A texturizing agent optionally can be included in the depilatory composition. In some embodiments, the texturizing agent can be added to the resinous or resinous tackifying agent. In non-limiting examples, the texturizing agent can be an oil, an ester of a fatty acid, a wax, or combinations thereof. The oil can be a natural or synthetic oil, such as a paraffinic oil. The ester may be a known ester of fatty acid, where the acid can comprise from 12 to 30 carbon atoms, preferably from 14 to 20 carbon atoms.

The wax may be present in the composition as a texturizing agent as well as a viscosity adjuster. Suitable waxes are natural waxes such as beeswax, lanolin, and hydrocarbon waxes such as microcrystalline wax or paraffin wax. A mixture of two waxes can be preferred and can be a mixture of at least one natural wax and at least one synthetic wax, or a mixture of at least two synthetic waxes or a mixture of at least two natural waxes. A preferred mixture is a mixture of paraffin wax and microcrystalline wax, for example, combined in a ratio (paraffin wax to microcrystalline wax) of about 5:1 to about 1:5, preferably about 3:1 to about 1:2.

The overall amount of texturizing agent present in the depilatory composition can be about 7% to about 30% by weight, preferably about 15% to about 20% by weight, based on the total weight of the depilatory composition.

### Polymer.

A polymer suitable for use in the depilatory composition preferably can be a thermoplastic polymer or an elastomeric polymer. In some embodiments, the thermoplastic polymer can be a polyolefin such as polyethylene (PE). Polyolefins can have varying hardness (e.g. ASTM D5 penetration values at 25° C. of about 0.2 to about 8 dmm), varying drop point Mettler (e.g. about 90° C. to about 130° C.), and varying viscosities (e.g. about 100 to about 1000 cps measured at 140° C. with a Brookfield viscometer). It is understood that other thermoplastic polymers exhibiting similar characteristics likewise may be used. Polyalphaolefins (PAO), for example, can be particularly useful.

Various elastomeric polymers likewise may be useful, such as acrylic polymers and copolymers. For example, ethylene butyl acrylate (EBA) and like acrylate copolymers may be used. Further examples of suitable elastomeric polymers are styrene copolymers such as styrene-butadiene and styrene-isoprene copolymers, e.g. SIS, SBS, SIBS, SEBS, SEPS, SIS/SIBS, SEBS/SEPS, optionally grafted e.g. with maleic anhydride; ethylene-propylene copolymers; ethylene-propylene terpolymers; vinyl ethylene-acetate copolymers, acrylic polymers; butadiene, isoprene, isoprene-isobutene, isobutene and chloroprene elastomers; nitrile rubbers and polyester thermoplastic elastomers. Further details of such elastomers may be found in, for example, U.S. Pat. No. 5,154,919, which is incorporated herein by reference. A preferred composition is one in which the elastomer is selected from styrene-butadiene, styrene-isoprene copolymers, EVA and mixtures thereof.

The inclusion of the polymer or polymers can be beneficial to provide a desired viscosity profile. In particular, the composition of the invention has a viscosity profile that provides for ease of use of the composition. In some embodiments, the composition can exhibit a viscosity profile whereby only small increases in temperature can provide significant decreases in the viscosity. The viscosity is a plane-plane viscosity, with the following settings:
apparatus: Rheometer Discovery DHR-1 (TA Instruments)
operating conditions: oscillating mode
plane-plane gap 800 microns
sample is deposited at 100° C. with a spatula
sample is conditioned at 100° C. with a preshear of 2 minutes at 10 s⁻¹
temperature ramp from 100° C. to 10° C. at 3° C./min
frequency 10 s⁻¹.

The present composition can exhibit various textural improvements. In particular, the compositions can exhibit a feeling for the user of a creamy texture, which can be more pleasant for the user.

The total amount of the thermoplastic and/or elastomeric polymer present in the depilatory composition can be about 1% to about 15% by weight, preferably about 2% to about 6% by weight, based on the total weight of the depilatory composition. In certain embodiments, a combination of polymers is used with the total concentration of the combined polymers being within the above concentrations.

### Surfactant

Suitable surfactants for use in the present composition can include, for example, nonionic, anionic, cationic, amphoteric or zwitterionic surfactants or mixtures thereof. Examples of useful nonionic surfactants are alkoxylated fatty alcohols such as fatty alcohols containing from 8 to 18 carbon atoms, especially from 12 to 16 carbon atoms, with from 2 to 10 ethoxy groups, especially 3 to 7 ethoxy groups. Further examples of surfactants are alcohol-oil transesterified surfactants such as PEG hydrogenated oils, sorbitan fatty acid esters, polyethylene glycol sorbitan fatty acid esters, sterol and sterol derivatives.

In some embodiments, useful surfactants include polyethylene glycol sorbitan fatty acid esters, also known as polysorbate. A preferred surfactant is polysorbate 80.

The total amount of surfactant in the depilatory composition can be about 1% to about 20%, preferably about 2% to about 8% by weight, based on the total weight of the depilatory composition.

### Water

Water is also present in the composition. Water allows a better temperature profile, including a lower application temperature. The total amount of water in the composition can be about 3% to about 15% by weight, preferably about 3% to about 6% by weight, based on the total weight of the depilatory composition.

### Heating Improving Agent

One or more metal salts can be included in the composition and can particularly act as a heat improving agent. The metal salt preferably can be a microwave conducting agent. The metal salt can be useful to provide a shorter heating time for the depilatory composition from a storage temperature to an application temperature.

The metal salt can be a salt of a metal which can be an alkaline or alkaline earth metal. Exemplary metals are Li, Na, K, Mg, Ca. The counter-ion can be a halide or a polyatomic oxygen-containing negative ion. Exemplary counter-ions are chloride, bromide, sulfate, nitrate, phosphate, and carbonate.

Exemplary metal salts are halides of alkaline metals such as LiCl, LiBr, NaCl, NaBr, KCl, KBr. NaCl which is readily available and can be preferred. The salt can be added to the water at any concentration up to saturation. Preferably the salt, especially NaCl, is used at a concentration that is between saturation and half the saturation concentration. Mixtures of metal salts are also envisaged.

The amount of metal salt as heating improving agent can be about 0.1% to about 10% by weight, preferably about 0.25% to about 5% by weight, most preferably about 0.5 to about 2% by weight, based on the total weight of the depilatory composition.

Other Ingredients Other ingredients commonly used in depilatories found in the prior art can also be present. For example, perfumes and essential oils can be incorporated if it is so wished. Preservatives, coloring agents and the like can also be present, albeit this is generally not necessary. Minerals and metal oxides, such as ZnO, or any thermal interface material as disclosed in WO-A-2013/160690 may also be present in the composition. In addition, any one or more specific materials otherwise described herein may be expressly excluded from compositions according to some embodiments of the present disclosure. Further, one or more specific materials not described herein may also be expressly excluded from embodiments of the present compositions. For example, calcium carbonate, glycerin, and mica all have previously been utilized in forming depilatory compositions, and any one or more of calcium carbonate, glycerin, and mica specifically may be excluded from embodiments of the present compositions.

### Method of Making.

The compositions of the present invention may be prepared by a variety of methods. For example, the rosinous or resinous tackifying agent may be melted together with the wax and the polymer if present. The final temperature should preferably not exceed 95° C. Separately water may be mixed with the surfactant, and then added to the molten rosinous or resinous tackifying agent and mixed in until the composition is homogeneous. Both phases can have the same or a similar temperature when they are mixed, or the water portion may be at room temperature or some other temperature when it is added to the rosinous or resinous portion. The mixture may then be cooled down to room temperature. Other ingredients may be added either to the rosinous or resinous portion or the water portion, depending on their properties and solubilities. Typically the salt such as NaCl is added to the water.

### Container.

The container may be of a traditional design such as is presently found in the art. In this regard, the invention provides a depilatory kit comprising a container containing an amount of a depilatory composition as otherwise described herein. Specifically, the depilatory kit can comprise a microwaveable container containing an amount of the depilatory composition suitable for one or more uses by a consumer. The microwaveable container preferably is formed of any material that is safe for heating in a microwave and that does not melt at a temperature less than an application temperature of the depilatory composition—e.g., preferably not melting at a temperature that is less than about 70° C. or less than about 100° C. In some embodiments, the depilatory kit can comprise an applicator and/or one or more composition removal strips (strips for removal of applied depilatory composition)—e.g., fabric strips.

### Method of Using.

When used, the composition of the present invention may simply be heated, such as to a temperature of about 35° C. or greater (e.g., up to a maximum of about 60° C. or about 70° C.). Heating may be carried out by any means such as by placing a container comprising the composition in a bath of hot water, such as a saucepan of boiling water. Most desirably, however, the composition of the present invention can be heated in an oven or a microwave oven. The length of time that the composition must be heated will depend on a number of factors, including the nature of composition itself, the quantity of composition to be heated, the nature of the container in which it is held and the power of the oven. Heating times in a microwave oven at maximum power (e.g. 900 W or 1000 W) can range from 30 seconds to 3 minutes, depending on the quantity being heated. Suitable heating instructions may be provided in association with the container containing the composition. Once the composition has been warmed, it may be stirred, if desired, in particular to ensure a more uniform temperature throughout the composition.

The depilatory may then be applied by a user to the skin, for example using a spatula or applicator. The application temperature may vary based upon the specific composition. In some embodiments, the application temperature can be about 35° C. to about 60° C., preferably in a range of about 45° C. to about 55° C. or about 35° C. to about 45° C. The composition is then allowed to set on the skin and hold the hair, or a strip of material such as a fabric can be applied over the composition and allowed to adhere thereto. The composition, or the composition and the fabric together, but preferably the composition without any fabric, can then be pulled or peeled from the skin in order to remove the hair therefrom. There is no minimum length for the hair in order for the hair to be removed satisfactorily.

The inventive composition demonstrated a creamy texture at the typical use temperatures disclosed herein.

The compositions of the present invention are such that residues are removable from the user's skin by washing with an oily material such as (paraffin) oil.

Unless specified to the contrary, all percentages are by weight based on the total weight of the depilatory composition.

### EXAMPLES

The following further examples illustrate the invention without limiting it.

### Example 1

The following composition for a 400 g weight is manufactured.

| Phase | Component | % |
|---|---|---|
| A | Glyceryl rosinate | 57-67 |
| A | Methyl hydrogenated rosinate | 4-8 |
| A | Paraffin (wax) | 6-15 |
| A | Polyethylene | 2-6 |
| A | Microcrystalline wax | 4-10 |
| B | Polysorbate 80 | 2-8 |
| B | Water | 2-6 |
| B | ZnO | 0.5-2 |
| B | NaCl | 0.5-2 |
| | | 100 |

### Example 2

The following composition for a 400 g weight is manufactured.

| Phase | Component | % |
|---|---|---|
| A | Glyceryl rosinate | 57-67 |
| A | Methyl hydrogenated rosinate | 4-8 |
| A | Paraffin (wax) | 6-15 |
| A | Polyethylene and EBA | 2-6 |
| A | Microcrystalline wax | 4-10 |
| B | Polysorbate 80 | 2-8 |
| B | Water | 2-6 |
| B | NaCl | 0.5-2 |
| | | 100 |

For preparation, an oil bath is heated and the phase A components are heated to 90° C. until complete homogenization by mixing using a lump breaker after melting. The ingredients in phase B are then mixed, and after mixing are added to phase A, homogenized thoroughly, and cooled to room temperature.

The composition of example 1 was tested for hair removal.

It was applied at a temperature of 45-55° C., and pro vided stiff strips once solidified on the user's skin.

The composition was tested for its ability to be heated in a microwave oven. The composition was placed in a 1000 W oven for a duration of 50 seconds with the container being placed (i) off center and (ii) at the center. The temperature was measured at one point in the center of the composition, half way down the pot and at one point at the edge of the composition, half way down the pot.

When placed off center, the composition of the invention exhibited a temperature of about 53° C., with a very low heterogeneity, i.e. minimal difference in temperatures between the two measurement points. When placed centrally, the composition of the invention exhibited a maximum temperature of 50° C., with a low heterogeneity. Hence the position of the tub with the composition (center or off-center) has no significant effect on the heating.

No rest time was needed before applying the composition of the invention.

The oxidative induction time for the composition of example 1 was higher than 2 minutes, evidencing stability. Further the viscosity profile of the composition of example 1 was such that:
the softening point was about 75-80° C.
the viscosity at 50° C. was lower than 100 Pa·s.

A clinical study was performed in order to evaluate the hair removal efficacy, the cutaneous tolerance, organoleptic characteristics.

The assessment criteria were hair removal efficacy (quantitatively counting of hair numbers on skin before and after depilation), subjective evaluation (questionnaire), cutaneous tolerance (clinical examination and collection of unpleasant sensations). The reference product was the product Monoi® of the applicant.

41 patients were enrolled for waxing on the body, 24 for waxing on the face. The average age was 36±2 years (between 18 and 54).

The main inclusion criteria were:
Sex: female.
Age between 18 and 55
Subject being used to waxing the zones of the study.
Subject with numerous hair on the zones of the study.
Subject having removed hair by the same method two weeks before enrollment for 2 mm of hair length.
Subject with sensitive skin on the face and the body (50%).
Subject with other type of skin on the face and the body (50%).

Under these study conditions, the composition of the invention was as effective on the hair removal as the Monoi® reference, with no significant difference concerning the counting of hairs on the armpits, half-legs, eyebrows and upper lip, is better-tolerated than reference product on the body and the face, and is appreciated by the subjects and by the beauticians for its characteristics and for its efficacy, for its convenient temperature of application, its creamy texture and its ease of homogenization

1. A depilatory composition comprising:
at least one rosinous or resinous tackifying agent;
at least one surfactant;
at least one metal salt; and
water.

2. The depilatory composition of claim 1, wherein the metal salt is selected from the group consisting of halides or polyatomic oxygen-containing negative ions of alkaline or alkaline-earth metals.

3. The depilatory composition of claim 2, wherein the metal salt is NaCl.

4. The depilatory composition of claim 1, comprising about 0.1% to about 10% by weight of the metal salt, based on the total weight of the depilatory composition.

5. The depilatory composition of claim 1, further comprising at least one texturizing agent.

6. The depilatory composition of claim 5, wherein the at least one texturizing agent is selected from the group consisting of an oil, a fatty acid ester, a wax, and mixtures thereof.

7. The depilatory composition of claim 6, wherein the at least one texturizing agent is a wax selected from the group consisting of paraffin, beeswax, microcrystalline wax, and mixtures thereof.

8. The depilatory composition of claim 7, wherein the texturizing agent comprises paraffin and microcrystalline wax.

9. The depilatory composition of claim 1, further comprising at least one thermoplastic polymer or elastomeric polymer.

10. The depilatory composition of claim 9, comprising one or both of polyethylene and ethylene butyl acrylate.

11. The depilatory composition of claim 1, wherein the depilatory composition comprises, based on the total weight of the depilatory composition:
about 50% to about 90% by weight of the at least one rosinous or resinous tackifying agent;
about 7% to about 30% by weight of at least one texturizing agent;
about 1% to about 15% by weight of at least one thermoplastic polymer or elastomeric polymer;
about 1% to about 20% by weight of the at least one surfactant;
about 0.1% to about 10% by weight of the at least one metal salt; and
about 3% to about 15% by weight of the water.

12. The depilatory composition of claim 1, wherein the depilatory composition comprises, based on the total weight of the depilatory composition:
about 60% to about 80% by weight of the at least one rosinous or resinous tackifying agent;
about 15% to about 20% by weight of at least one texturizing agent;
about 2% to about 6% by weight of at least one thermoplastic polymer or elastomeric polymer;
about 2% to about 8% by weight of the at least one surfactant;
about 0.1% to about 10% by weight of the at least one metal salt; and
about 3% to about 6% by weight of the water.

13. The depilatory composition of claim 1, wherein the rosinous or resinous tackifying agent comprises glyceryl rosinate and methyl hydrogenated rosinate.

14. The depilatory composition of claim 1, wherein the surfactant is a polysorbate.

15. The depilatory composition of claim 1, wherein the depilatory composition comprises, based on the total weight of the depilatory composition:
about 60% to about 80% by weight of glyceryl rosinate and methyl hydrogenated rosinate;
about 15% to about 20% by weight of paraffin and microcrystalline wax;
about 2% to about 6% by weight of polyethylene and ethylene butyl acrylate;
about 2% to about 8% by weight of a polysorbate;
about 0.5% to about 2% by weight of NaCl; and
about 3% to about 6% by weight of water.

16. A depilatory kit comprising a microwaveable container containing an amount of the depilatory composition of claim 1.

17. The depilatory kit of claim 16, further comprising an applicator or a plurality of composition removal strips.

18. A method for removing hair from skin which comprises:
(a) heating a composition according to claim 1 to a temperature of about 35° C. or greater;
(b) applying the heated composition to the skin;
(c) allowing said composition to set and hold said hair or applying a strip of material over said composition and allowing said strip to adhere thereto; and
(d) removing said composition and hair by peeling said composition or said material and said composition, from the skin.

19. The method of claim 18, wherein said heating comprises heating the composition by microwaves.
